(19) United States
(12) Patent Application Publication
(10) Pub. No.: US 2012/0225107 A1
(43) Pub. Date: Sep. 6, 2012

(54) STABLE SKIN AND SCAR TREATMENT COMPOSITION

(76) Inventor: Caridad HECHAVARRIA, Branford, CT (US)

(21) Appl. No.: 13/410,745

(22) Filed: Mar. 2, 2012

Related U.S. Application Data
(60) Provisional application No. 61/449,447, filed on Mar. 4, 2011.

(51) Int. Cl. A61K 9/14; A61K 8/67; A61K 38/16; A61K 8/64; A61Q 19/00; A61P 29/00; A61Q 19/02; A61P 17/18; A61P 31/10; A61P 31/04; A61P 17/10; A61K 8/02; A61P 17/02

(52) U.S. Cl. 424/401; 424/400; 424/62; 514/18.6; 514/18.8

(57) ABSTRACT

Provided are dermatological compositions for skin and scar treatment, methods of making the compositions and methods of applying the compositions, which result in improved skin and scar whitening performance. In one form, the dermatological composition for skin and scar treatment includes 33 to 60 wt. % of one or more hydrophilic benefiting ingredients, and 40 to 60 wt. % of an anhydrous hydrophilic/amphiphatic base comprising 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS). The one or more hydrophilic benefiting ingredients may include skin whitening agents, antioxidants, extracts, antifungal/antibacterial/anti-acne agents, polypeptides, anti-inflammatory agents, keratolytic agents, amino acids and combinations thereof.

### CROSS-REFERENCE TO RELATED APPLICATIONS

This is a non-provisional application that claims priority to U.S. Provisional Application No. 61/449,447 filed on Mar. 4, 2011, herein incorporated by reference in its entirety.

### FIELD

The present disclosure relates to the field of skin and scar treatment. It more particularly relates to dermatological compositions for skin and scar treatment, methods of making the compositions and methods of treating skin and scars that result in improved skin and scar whitening performance.

### BACKGROUND

Many topical dermatological products have been developed with L-ascorbic acid, ascorbate derivatives such as sodium ascorbate, alpha hydroxyl acids such as glycolic acid and malic acid and other organic acids to improve skin appearance by their cell desquamation property; and skin lighteners such as hydroquinone, arbutin, kojic, and magnesium ascorbic phosphate acid for their skin lightening properties.

Presently, many of the commercial cosmetic skin desquamation and skin lightener dermatological products contain insufficient amounts of L-ascorbic acid because of solubility limitations and degradation of the active ingredients. These products predominately contain water based delivery systems, which limit the quantity of L-ascorbic acid, other forms of Vitamin C and other benefiting ingredients which may be contained in the compositions due to solubility limits.

Other desquamation or skin peeling agents used such trichloroacetic acid are aggressive and result in an inflammatory response to the subject for treatment which is not desirable. Exfoliating agents, such as retinoids (e.g., tretinoin, retinol and retinal), carboxylic acids including alpha.-hydroxy acids (e.g., lactic acid, glycolic acid), beta.-hydroxy acids (e.g. salicylic acid), alpha-keto acids, acetic acid and, salicylic acid, alpha-hydroxy decanoic acid, alpha.-hydroxy octanoic acid, gluconolactone, oxalic acid, malic acid, tartaric acid, mandelic acid, benzylic acid, gluconic acid, among others may also increase the skin's sensitivity to environmental conditions such as sunlight, wind, cold temperature and dry air, or may exacerbate the irritation attributable to a pre-existing skin disease. The skin lightener compositions containing tyrosinase inhibitors such as hydroquinone are also of limited efficacy due to allowable percent levels (2.0 wt. %) in cosmetic products that are considered safe.

Hence, a need exists for dermatological compositions for skin and scar treatment that allow for a significantly higher concentration of L-ascorbic acid, other forms of Vitamin C, and other benefiting ingredients that will not be subject to the solubility and degradation constraints currently present in the prior art dermatological compositions.

### SUMMARY

According to the present disclosure, an advantageous dermatological composition for skin and scar treatment comprises: 33 to 60 wt. % of one or more hydrophilic benefiting ingredients; to 60 wt. % of an anhydrous hydrophilic/amphiphatic base comprising 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS); and wherein the composition includes less than 5 wt. % water; wherein the one or more hydrophilic benefiting ingredients are in a dry solid form at room temperature with a particle size ranging from 2 to 12μ in diameter; and wherein the anhydrous hydrophilic/amphiphatic base provides for time-release of the one or more hydrophilic benefiting ingredients after topical application to the skin.

A further aspect of the present disclosure relates to an advantageous method of making a dermatological composition for skin and scar treatment comprising: providing components to make a composition comprising 33 to 60 wt. % of one or more hydrophilic benefiting ingredients and 40 to 60 wt. % of an anhydrous hydrophilic/amphiphatic base; grinding and then mixing the one or hydrophilic benefiting ingredients in a dry solid form at room temperature to a particle size ranging from 2 to 12μ; making the anhydrous hydrophilic/amphiphatic base by combining 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS) at a temperature ranging from 40 to 60° C.; mixing the ground and mixed one or more hydrophilic benefiting ingredients into the anhydrous hydrophilic/amphiphatic base at a sufficient shear rate, temperature and time to form a homogenous mixture of the one or more hydrophilic benefiting ingredients, and the anhydrous hydrophilic/amphiphatic base; forming the homogenous mixture into the form of a cake, a stick, a hot pour or a cream; and cooling the formed homogenous mixture to room temperature to form a dermatological composition for topical application to skin; wherein the composition includes less than 5 wt. % water; and wherein the anhydrous hydrophilic/amphiphatic base provides for time-release of the one or more hydrophilic benefiting ingredients after topical application to the skin.

Another aspect of the present disclosure relates to an advantageous method of treating skin and scars comprising: providing a dermatological composition including 33 to 60 wt. % of one or more hydrophilic benefiting ingredients, and 40 to 60 wt. % of an anhydrous hydrophilic/amphiphatic base comprising 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS); and topically applying the dermatological composition to a skin surface area for treatment in a form chosen from a stick, a cream, a cake or a hot pour; wherein the composition includes less than 5 wt. % water; wherein the one or more hydrophilic benefiting ingredients are in a dry solid form at room temperature with a particle size ranging from 2 to 12μ in diameter; and wherein the anhydrous hydrophilic/amphiphatic base provides for time-release of the one or more hydrophilic benefiting ingredients after topically applying the dermatological composition to the skin.

Still another aspect of the present disclosure relates to an advantageous dermatological composition for skin and scar treatment comprising: 33 to 60 wt. % of one or more hydrophilic benefiting ingredients, and 40 to 60 wt. % of an anhydrous hydrophilic/amphiphatic base comprising one or more water soluble polyols, and wherein the composition includes less than 5 wt. % water, wherein the one or more hydrophilic benefiting ingredients are in a dry solid form at room temperature with a particle size ranging from 2 to 12μ in diameter, and wherein the anhydrous hydrophilic/amphiphatic base provides for time-release of the one or more hydrophilic benefiting ingredients after topical application to the skin.

Yet still another aspect of the present disclosure relates to an advantageous method of making a dermatological composition for skin and scar treatment comprising: providing components to make a composition comprising 33 to 60 wt. % of one or more hydrophilic benefiting ingredients, and 40 to 60 wt. % of an anhydrous hydrophilic/amphiphatic base, grinding and then mixing the one or hydrophilic benefiting ingredients in dry solid form at room temperature to a particle size ranging from 2 to 12μ, making the anhydrous hydrophilic/amphiphatic base by combining one or more water soluble polyols at a temperature ranging from 40 to 60° C., mixing the ground and mixed one or more hydrophilic benefiting ingredients into the anhydrous hydrophilic/amphiphatic base at a sufficient shear rate, temperature and time to form a homogenous mixture of the one or more hydrophilic benefiting ingredients, and the anhydrous hydrophilic/amphiphatic base, forming the homogenous mixture into the form of a cake, a stick, a hot pour or a cream, and cooling the formed homogenous mixture to room temperature to form a dermatological composition for topical application to skin, wherein the composition includes less than 5 wt. % water, and wherein the anhydrous hydrophilic/amphiphatic base provides for time-release of the one or more hydrophilic benefiting ingredients after topical application to the skin.

These and other features and attributes of the disclosed dermatological compositions for skin and scar treatment and methods of making and using such compositions and their advantageous applications and/or uses will be apparent from the detailed description which follows, particularly when read in conjunction with the figures appended hereto.

### BRIEF DESCRIPTION OF DRAWINGS

To assist those of ordinary skill in the relevant art in making and using the subject matter hereof, reference is made to the appended drawings, wherein:

FIG. 1 depicts an exemplary schematic of the steps in making the dermatological compositions for skin and scar treatment disclosed herein.

### DETAILED DESCRIPTION

All numerical values within the detailed description and the claims herein are modified by “about” or “approximately” the indicated value, and take into account experimental error and variations that would be expected by a person having ordinary skill in the art.

Overview:

The present disclosure provides novel dermatological compositions for skin and scar treatment. The compositions are distinguishable over the prior art in allowing for higher levels or loadings of one or more benefiting ingredients in the compositions through the use of an anhydrous hydrophilic delivery system as opposed to a water based delivery system that is currently found in the prior art. The higher levels or loadings of one or more benefiting ingredients in the dermatological compositions provides for advantageous performance, which include, inter alia, improved skin whitening, collagen formation and lightening of scar tissue with time. The higher levels or loadings of one or more benefiting ingredients in the dermatological compositions disclosed herein also allows for more effective release to the skin of other active agents that may be included in the composition. The compositions are also stable under normal use conditions and withstand high relative humidity.

L-ascorbic acid is known for its skin lightening property, however in conventional water based delivery systems, its efficacy it limited by limitations in loading level. Even at high loading levels in water based delivery systems, L-ascorbic acid causes skin irritation and worsens scar/damaged areas of the skin. The applicants have unexpectedly discovered that that the use of L-ascorbic acid and other benefiting agents when used in an anhydrous hydrophilic/amphiphatic base disclosed herein can be loaded at higher levels and will not cause skin irritation, but will ameliorate scar tissue. The anhydrous hydrophilic/amphiphatic base formulation of the present disclosure allows the L-ascorbic acid to penetrate the skin overtime and promote collagen formation and the lightening of the scar tissue.

The anhydrous hydrophilic/amphiphatic base disclosed herein (topical cosmetic carrier) is a material or mixture of materials, which is compatible with the active/benefiting agents disclosed herein (and supplemental active materials, if included), is non-irritating when applied to the skin, provides cosmetic benefits, and may aid penetration of the active/benefiting agents into the skin. It also helps provide for a composition that is stable under normal use conditions and can withstand high relative humidity.

The dermatological compositions disclosed herein, although including one or more hydrophilic benefiting ingredients (also referred to as active ingredients) and an anhydrous hydrophilic/amphiphatic base, may also contain water at less than 5 wt. %, or less than 4 wt. %, or less than 3 wt. %, or less than 2 wt. %, or less than 1 wt. %, or less than 0.5 wt. % of the total composition. The one or more hydrophilic benefiting ingredients are in a dry solid form and for the purposes of the current disclosure are referred to as “Phase A.” The anhydrous hydrophilic/amphiphatic base may be in a solid, semi-solid or liquid form and for the purposes of the current disclosure are referred to as “Phase B.” The one or more hydrophilic benefiting ingredients may not be in a liquid form in order to achieve the advantageous benefits of the skin and scar treatment compositions disclosed herein.

The dermatological compositions for skin and scar treatment disclosed herein advantageously include ultrafine Ascorbic acid (average particle size in the 2-12 micron range) as one of the hydrophilic benefiting agents in the formulation. The one or more hydrophilic benefiting ingredients used in the formulation are also advantageously in a dry form. When two or more hydrophilic benefiting ingredients are used in the formulation, they are advantageously thoroughly mixed together prior to combining them with the anhydrous hydrophilic/amphiphatic base. The Applicants have unexpectedly and surprisingly discovered that the one or more hydrophilic benefiting ingredients when used in the anhydrous hydrophilic/amphiphatic base disclosed herein provides for improved skin and scar whitening performance when the following conditions are satisfied: 1) the benefiting ingredients are used in their dry form, 2) the benefiting ingredients have an ultrafine particle size in the range of 2 to 12 microns, and 3) the benefiting ingredients are thoroughly mixed together prior to being combined with the anhydrous hydrophilic/amphiphatic base.

In order to obtain a particle size of the benefiting ingredients disclosed herein, they may be ground in, for example, a pulverizer or a jet mill, prior to being incorporated into the anhydrous hydrophilic/amphiphatic base. One particularly advantageous benefiting ingredients in the compositions disclosed herein is Ascorbic Acid. In addition, when the three above conditions are satisfied, the dermatological compositions for skin and scar treatment disclosed herein are commercially acceptable and stable formulations (stable over a 3 month period at 40° C.). When the three above conditions are not satisfied, the dermatological compositions for skin and scar treatment disclosed herein are not commercially acceptable and are unstable. The commercially stable compositions are easily applied to the skin surface and provide a homogeneous delivery of the skin benefiting agents to the skin and/or scar area for whitening.

### Exemplary Dermatological Composition Embodiments

In one exemplary form, the dermatological composition for skin and scar treatment of the present disclosure includes 33 to 60 wt. % of one or more hydrophilic benefiting ingredients, and 40 to 60 wt. % of an anhydrous hydrophilic/amphiphatic base. The anhydrous hydrophilic/amphiphatic base includes 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS).

In another exemplary form, the dermatological composition for skin and scar treatment includes 33 to 60 wt. % of three or more hydrophilic benefiting ingredients including L-ascorbic acid at from 30 to 59.5 wt. %, Lactobacillus Ferment Lysate Filtrate at from 0.1 to 3.0 wt. %, and azelaic acid at from 0.5 to 10.0 wt. %, and 40 to 60 wt. % of an anhydrous hydrophilic/amphiphatic base. The anhydrous hydrophilic/amphiphatic base includes 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS).

In yet another exemplary form, the dermatological compositions disclosed herein may include ascorbic acid, ascorbic acid derivatives and/or extracts containing ascorbic acid in a substantially anhydrous hydrophilic/amphiphatic base (containing no or low levels of water, that is water at less than 5 wt. %, or less than 4 wt. %, or less than 3 wt. %, or less than 2 wt. %, or less than 1 wt. %, or less than 0.5 wt. % of the total composition). In this form, the anhydrous hydrophilic/amphiphatic base includes 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS).

In still yet another exemplary form, the dermatological composition for skin and scar treatment includes 33 to 60 wt. % of one or more hydrophilic benefiting ingredients, and 40 to 60 wt. % of an anhydrous hydrophilic/amphiphatic base comprising one or more water soluble polyols. In this form, the one or more water soluble polyols are chosen from glycerol, sorbitol, glucose, fructose, lactose, propylene glycol, polyethylene glycol, cetyl alcohol, stearyl alcohol, ethoxydiglycol, dexapanthenol, phenoxyethanol, and tocophersolan (TPGS).

In each of the exemplary composition embodiments described above, the composition may include less than 5 wt. % water and the one or more hydrophilic benefiting ingredients are in a dry solid form at room temperature with a particle size ranging from 2 to 12μ in diameter. Also in each of the exemplary composition embodiments described above, the anhydrous hydrophilic/amphiphatic base may provide for time-release of the one or more hydrophilic benefiting ingredients after topical application to the skin.

The anhydrous hydrophilic/amphiphatic base provides a unique cosmetically acceptable topical vehicle which protects the ascorbic acid or its derivatives from degradation, instability, loss of potency and color change. The anhydrous hydrophilic/amphiphatic base may be a polyol and fatty alcohol/acid carrier system. The anhydrous hydrophilic/amphiphatic base disclosed above provides for time-release of the one or more hydrophilic benefiting ingredients after topical application to the skin.

The anhydrous hydrophilic/amphiphatic base may also optionally include fillers/extenders. Non-limiting exemplary fillers/extenders that may be added to the anhydrous hydrophilic/amphiphatic base include spherical silica, polyamides (Nylon), mica, talc, polyethylene, polytetrafluoroethylene (PTFE), clay, polyester and combinations thereof. These fillers/extenders are typically in solid form. The fillers/extenders may be present in the dermatological composition for skin and scar treatment disclosed herein in the range of 1 to 20 wt. %, or 2 to 15 wt. %, or 5 to 10 wt. % based on the total weight of the composition.

The hydrophilic active or benefiting ingredients present in the anhydrous hydrophilic/amphiphatic base are present in a range of from 33 to 60 weight percent, and more preferably 40 to 50 weight percent. Alternatively, the one or more hydrophilic benefiting ingredients disclosed herein may constitute from 33 to 60 wt. %, or 35 to 57 wt. %, or 40 to 60 wt. %, or 45 to 55 wt. %, or 50 wt. % of the dermatological compositions disclosed herein. The hydrophilic benefiting ingredient is inherently unstable in water and is also sufficiently soluble in water. The hydrophilic benefiting ingredients disclosed herein not only provide specific benefiting effects to the skin, but also assist in the efficacy of other active agents disclosed below.

The one or more hydrophilic benefiting ingredients disclosed herein are advantageously in a dry solid form at room temperature and in the form of ultrafine particles with a particle size ranging from 2 to 12μ in diameter, or 2 to 8μ in diameter, or 4 to 8μ in diameter, or 5 to 7μ in diameter. At larger particle sizes for the one or more hydrophilic benefiting ingredients in solid form, they become abrasive.

The dry solid form of the one or more hydrophilic benefiting ingredients assists with solubility and also with release from the composition to the skin interface.

The dermatological compositions for skin and scar treatment of the present disclosure may be formed into a solid form, such as a stick, a cake, a hot pour, or other suitable shape for solid application. These solid forms may have a melting point of from 40 to 70° C., or 45 to 60° C., or 50 to 55° C., or 52 to 54° C. One non-limiting exemplary solid form (hot pour for molded stick or cake) includes the following ingredients: ultrafine ascorbic acid at 40-50 wt. %, arbutin at 2.0-5.0 wt. %, propylene glycol at 18.0-22.0 wt. %, cetyl alcohol at 8.0-11.0 wt. %, azelaic acid at 1.0 4.0 wt. %, ethoxydiglycol at 3.0 5.0 wt. %, polyethylene glycol 1000 at 5.0-8.0 wt %, phenoxyethanol at 0.5-1.0 wt %, dexapanthenol at 0.5-2.0 wt. %, and tocopherolsolan at 0.5-2.0%, ac dermapeptide lightening at 0.5-1.5 wt %. The ingredients are customized to deliver a thick paste that can be hot poured into an appropriate package at a specific temperature (typically in the range of 40 to 70 degrees Celsius) to form a molded stick, compact, cake or other solid form.

The dermatological compositions for skin and scar treatment of the present disclosure may be formed into a liquid form, such as a gel or a cream for topical application to skin. These liquid forms may have a viscosity at room temperature ranging from 5,000 to 300,000 cP, or 10,000 to 200,000 cP, or 20,000 to 100,000 cP, or 40,000 to 80,000 cP, or 55,000 to 65,000 cP as measured with a Brookfield viscometer. One non-limiting exemplary cream form includes the following ingredients: ultrafine ascorbic acid at 40-50 wt. %, arbutin at 2.0-10.0 wt. %, propylene glycol at 22.0-26.0 wt. %, ethoxydiglycol at 3.0-5.0 wt. %, cetyl alcohol at 5.0-8.0 wt. %, polyethylene glycol 1000 at 4.0-5.0 wt %, phenoxyethanol at 0.5-1.0 wt. %, dexapenthanol at 0.5-2.0 wt. %, and ac dermapeptide lightening (active concepts) at 0.5-2.0 wt. %. a second non-limiting exemplary cream form includes the following ingredients: ultrafine ascorbic acid at 40-50 wt. %, glycine at 2.0-6.0 wt. %, propylene glycol at 22.0-26.0 wt. %, ethoxydiglycol at 3.0-5.0 wt. %, cetyl alcohol at 4.0-6.0 wt. %, polyethylene glycol 1000 at 4.0-5.0 wt. %, phenoxyethanol at 0.5-1.0 wt. %, dexapanthenol at 0.5-2.0 wt. %, and ac dermapeptide lightening (active concepts). The ingredients are customized to deliver a cream consistency to be filled into an appropriate package at room temperature, such as a tube, jar or other container.

In addition to the one or more hydrophilic benefiting ingredients disclosed herein, the dermatological compositions for skin and scar treatment of the present disclosure may also include water soluble antimicrobials and other water soluble (hydrophilic) active agents, which are described in further detail below. If non-water soluble active agents are included in the compositions disclosed herein, they must be present at sufficiently low levels. If hydrophobic active agents are included in the compositions disclosed herein, they must be pre-processed with suitable surfactants or emulsifying agents to be in a micellar form to be available for delivery to the skin.

The one or more hydrophilic benefiting ingredients disclosed herein may include skin whitening agents, antioxidants, extracts, antifungal/antibacterial/anti-acne agents, polypeptides, anti-inflammatory agents, keratolytic agents, amino acids and combinations thereof, which are described in more detail below.

Whitening Agents:

The whitening agents suitable as one or more of the hydrophilic benefiting ingredients disclosed herein, include, but are not limited to, Vitamin C, hydroquinone, carbimide peroxide, azelaic acid, cysteine, cysteine HCl, butylhydroquinone, dicetyl, disodium ascorbyl sulfate, erythorbic acid, sugar ester derivatives of Vitamin C, ethyl ferulate, ferulic acid, gallic acid esters, kojic acid, beta & alpha arbutin, Tyrostat (rumex occidentalis extract), cashew fruit extract, curcuma extract, acerola extract, white mulberry moraceae extracts, licorice extract, propyl resorcinol, licorice extract/glabridin, Lactobacillus Ferment Lysate Filtrate, tetrahydrocurcuminoid (THC), and combinations thereof. Non-limiting exemplary forms of Vitamin C include magnesium ascorbyl phosphate, L-ascorbic acid, ascorbyl glycoside, ascorbic acid, and combinations thereof.

In one advantageous form, the Vitamin C in the dermatological compositions disclosed herein ranges from 40 to 55 wt. %, or 42 to 52 wt. %, or 45 to 50 wt. %, or 50 wt. % of the composition. The dermatological compositions disclosed herein may also include azelaic acid ranging from 0.5 to 10 wt. %, or 1 to 9 wt. %, or 2 to 8 wt. %, or 3 to 7 wt. % or 4 to 6 wt. % of the composition. The dermatological compositions disclosed herein may also include Lactobacillus Ferment Lysate Filtrate ranging from 0.1 to 3.0 wt. %, or 0.2 to 2.8 wt. %, or 0.4 to 2.6 wt. %, or 0.8 to 2.4 wt. %, or 1.0 to 2.0 wt. %, or 1.4 to 1.6 wt. %.

Antioxidants:

The antioxidants suitable as one or more of the hydrophilic benefiting ingredients disclosed herein, include, but are not limited to, green tea extracts, grape seed extracts, alpha-lipoic acid, potassium sulfite, propyl gallate, quinones, rosmarinic acid, sodium ascorbate, sodium erythorbate, sodium metabisulfite, sodium sulfite, superoxide dismutase, sorbityl furfural, tocopheryl succinate, genistein, quercetin, passiflora extract, chrysin, naringenin-7-glucoside, Vitamin C and combinations thereof.

Extracts:

The extracts suitable as one or more of the hydrophilic benefiting ingredients disclosed herein, include, but are not limited to, algae extract, aloe barbadensis extract, althea officinalis extract, birch (betula alba) bark extract, borage (borago officinalis) extract, eucalyptus globulus extract, evening primrose (oenothera biennis) extract, geranium maculatum extract, jasmine (jasminum officinale) extract, jojoba (buxus chinensis) oil, kelp, sage (salvia officinalis) extract, licorice extract, uva ursi extract, bearberry extract, rumex crispus extract, blackberry bark extract, gingko biloba extract, ginseng extract, cucumber extracts, willow bark extract and combinations thereof.

Antifungal/Antibacterial/Anti-Acne Agents:

The antifungal/antibacterial/anti-acne agents suitable as one or more of the hydrophilic benefiting ingredients disclosed herein, include, but are not limited to, cedar leaf extract, teat tree extract, citricidal, oleuropein, azelaic acid, Fluorometholone, benzoyl peroxide, Tretinoin, Retinol, Adapalene, Isotretinoin, Motretinide, Clindamycin, Erythromycin, Chloramphenicol, Meclocycline, Resorcinol, aluminium oxide, Dapsone, Vitamin A derivatives, and combinations thereof.

Polypeptides:

The polypeptides suitable as one or more of the hydrophilic benefiting ingredients disclosed herein, include, but are not limited to, glutathione (γ-glutamyl-cysteinyl-glycine), copper peptides, palmitoyl entapeptide-4(Matrixyl™) and combinations thereof.

Keratolytic Agents:

The keratolytic agents suitable as one or more of the hydrophilic benefiting ingredients disclosed herein, include, but are not limited to, Retin A, topical Fluorouracil, Occlusal-HP (Duoderm) and combinations thereof.

Anti-Inflammatory Agents:

The anti-inflammatory agents suitable as one or more of the hydrophilic benefiting ingredients disclosed herein, include, but are not limited to, water soluble salts of corticosteroids, noncorticosteroids and combinations thereof.

Amino Acids:

The amino acids suitable as one or more of the hydrophilic benefiting ingredients disclosed herein, include, but are not limited to, glycine, lysine, argenine, gluthathione, proline and combinations thereof.

Other Active Agents:

The dermatological compositions for skin and scar treatment of the present disclosure may also include one or more other active agents to give one or more advantageous properties to the compositions. Preferably these other active agents are hydrophilic in nature. If non-water soluble active agents are included in the compositions disclosed herein, they must be present at sufficiently low levels. If hydrophobic active agents are included in the compositions disclosed herein, they must be pre-processed with suitable surfactants or emulsifying agents to be in a micellar form to be available for delivery to the skin. These other active agents include, but are not limited to, humectants, and antimicrobial agents, which are described below.

Humectants:

The dermatological compositions for skin and scar treatment of the present disclosure may also include one or more humectants. The humectant serves to keep dermatological compositions from hardening upon exposure to air. The humectant, on a pure humectant basis, generally comprises from 0% to 30%, or 1% to 25%, or 2% to 20%, or 3% to 10%, or 4% to 8% by weight of the compositions herein. Exemplary non-limiting humectants include glycerin, sorbitol, xylitol, butylene glycol, polyethylene glycol, propylene glycol and combinations thereof.

Antimicrobial Agents:

The dermatological compositions of the present disclosure may also include other agents, such as antimicrobial agents. Included among such agents are copper bisglycinate, copper glysinate, zinc citrate, and zinc lactate. Other antimicrobial & anti-fungal agents, such as Tea tree extract, cedar leaf extract, citricidal, azelaic acid, resorcinol, Vitamin A derivatives, clindamycin, erythromycin, etc.

### Exemplary Method of Making Embodiments

In one exemplary form, a method of making a dermatological composition for skin and scar treatment includes the following steps: 1) providing components to make a composition comprising 33 to 60 wt. % of one or more hydrophilic benefiting ingredients, and 40 to 60 wt. % of an anhydrous hydrophilic/amphiphatic base, 2) grinding and then mixing the one or hydrophilic benefiting ingredients in solid form at room temperature to a particle size ranging from 2 to 12μ, 3) making the anhydrous hydrophilic/amphiphatic base by combining 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS) at a temperature ranging from 40 to 60° C., 4) mixing the ground one or more hydrophilic benefiting ingredients into the anhydrous hydrophilic/amphiphatic base at a sufficient shear rate, temperature and time to form a homogenous mixture of the one or more hydrophilic benefiting ingredients, and the anhydrous hydrophilic/amphiphatic base, 5) forming the homogenous mixture into the form of a cake, a stick, a hot pour or a cream, and finally 6) cooling the formed homogenous mixture to room temperature to form a dermatological composition for topical application to skin.

In another exemplary form, a method of making a dermatological composition for skin and scar treatment includes the following steps: 1) providing components to make a composition comprising 33 to 60 wt. % of one or more hydrophilic benefiting ingredients, and 40 to 60 wt. % of an anhydrous hydrophilic/amphiphatic base, 2) grinding and then mixing the one or hydrophilic benefiting ingredients in solid form at room temperature to a particle size ranging from 2 to 12μ, 3) making the anhydrous hydrophilic/amphiphatic base by combining one or more water soluble polyols at a temperature ranging from 40 to 60° C., 4) mixing the ground one or more hydrophilic benefiting ingredients into the anhydrous hydrophilic/amphiphatic base at a sufficient shear rate, temperature and time to form a homogenous mixture of the one or more hydrophilic benefiting ingredients, and the anhydrous hydrophilic/amphiphatic base, 5) forming the homogenous mixture into the form of a cake, a stick, a hot pour or a cream, and 6) cooling the formed homogenous mixture to room temperature to form a dermatological composition for topical application to skin.

In each of the exemplary method of making embodiments described above, the composition may include less than 5 wt. % water and the one or more hydrophilic benefiting ingredients are in a dry solid form at room temperature with a particle size ranging from 2 to 12μ in diameter. Also in each of the exemplary method of making embodiments described above, the anhydrous hydrophilic/amphiphatic base may provide for time-release of the one or more hydrophilic benefiting ingredients after topical application to the skin.

The mixing advantageously occurs in a homogenizer that is oil or water heated with temperature control. The mixing time, the mixing speed and the mixing temperature of the homogenizer may be independently controlled. The mixing and/or forming steps disclosed above may include also molding or extruding processes to form the solid form, such as a cake, a stick, or a hot pour. Alternatively, the forming step for the liquid form may include filling the homogenous mixture disclosed above into a tube for a cream or a gel at a temperature of room temperature to 45° C.

The sufficient shear rate for mixing the ground one or more hydrophilic benefiting ingredients into the anhydrous hydrophilic/amphiphatic base may occur at a shear rate for mixing in the homogenizer ranging from 2,000 to 16,000 revolutions per minute (rpm), or 3,000 to 10,000 rpm, or 3,000 to 6,000 rpm.

The sufficient temperature for mixing the ground one or more hydrophilic benefiting ingredients into the anhydrous hydrophilic/amphiphatic base in the homogenizer may occur at a temperature for mixing ranging from 22 to 60° C., or 30 to 55° C., or 35 to 50° C.

The sufficient time for mixing the ground one or more hydrophilic benefiting ingredients into the anhydrous hydrophilic/amphiphatic base in the homogenizer may occur for a time ranging from 2 to 60 minutes, or 5 to 50 minutes, or 10 to 30 minutes.

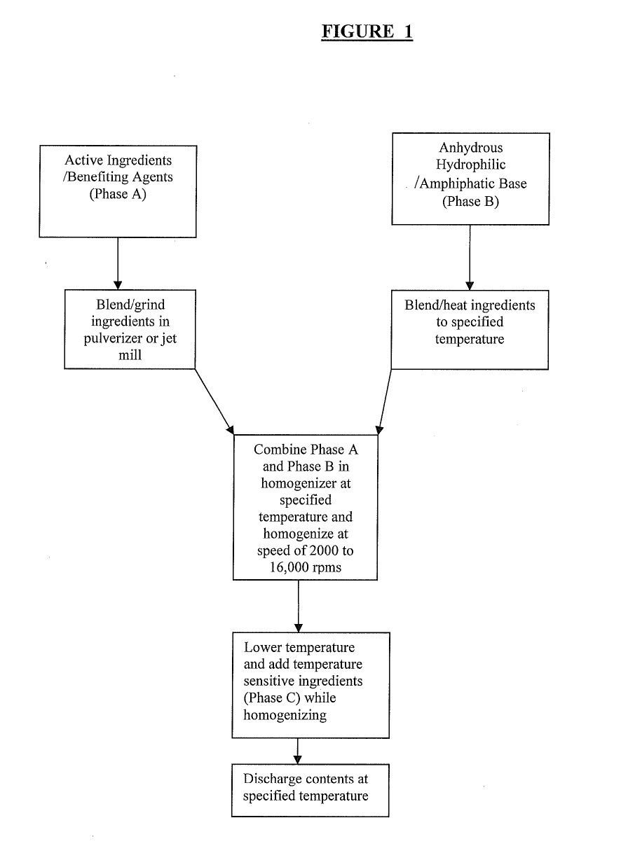

FIG. 1 is an exemplary process flow diagram that summarizes the steps in making the dermatological compositions for skin and scar treatment disclosed above.

Exemplary Method of Treating Skin:

In one exemplary form, a method of treating skin and scars includes the following steps: 1) providing a dermatological composition including 33 to 60 wt. % of one or more hydrophilic benefiting ingredients, and 40 to 60 wt. % of an anhydrous hydrophilic/amphiphatic base; and 2) topically applying the dermatological composition to a skin surface area for treatment in a form chosen from a stick, a cream, a cake or a hot pour. In one form, the anhydrous hydrophilic/amphiphatic base includes one or more polyols chosen from glycerol, sorbitol, glucose, fructose, lactose, propylene glycol, polyethylene glycol, cetyl alcohol, stearyl alcohol, ethoxydiglycol, dexapanthenol, phenoxyethanol, and tocophersolan (TPGS). In another form, the anhydrous hydrophilic/amphiphatic base includes 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS).

In each of the exemplary method of treating embodiments described above, the composition may include less than 5 wt. % water and the one or more hydrophilic benefiting ingredients are in a dry solid form at room temperature with a particle size ranging from 2 to 12μ in diameter. Also in each of the exemplary method of treating embodiments described above, the anhydrous hydrophilic/amphiphatic base may provide for time-release of the one or more hydrophilic benefiting ingredients after topical application to the skin.

The anhydrous hydrophilic/amphiphatic base provides for time-release of the one or more hydrophilic benefiting ingredients after topically applying the dermatological composition to the skin. The moisture from skin surface area for treatment may initiate the time-release of the one or more hydrophilic benefiting ingredients after topically applying the dermatological composition to the skin via a passive release mechanism. Alternatively, the skin surface area for treatment may be pre-wet prior to topically applying the dermatological composition. The pre-wet skin surface area for treatment initiates time-release of the one or more hydrophilic benefiting ingredients after topically applying the dermatological composition to the skin via an active release mechanism.

As disclosed above, prior to step of topically applying the dermatological composition to a skin surface area for treatment, the skin area for treatment may be pre-wet prior to application to help activate the release of the one or more hydrophilic benefiting ingredients in the composition to the skin interface for treatment. Alternatively, if the skin is not pre-wet, the moisture from the skin area for treatment may supply the necessary moisture to the dermatological composition for activation of the release of the one or more hydrophilic benefiting ingredients in the composition to the skin interface.

The method of treating skin and scars with the dermatological compositions disclosed herein may be repeated by topically applying step on at least a daily basis to the skin surface area for treatment. After topical application of the dermatological compositions disclosed herein with time, the skin surface area for treatment undergoes whitening, collagen formation and lightening of scar tissue with time.

### Other Alternative Embodiments

A dermatological composition for skin and scar treatment comprising 33 to 60 wt. % of one or more hydrophilic benefiting ingredients, and 40 to 60 wt. % of an anhydrous hydrophilic/amphiphatic base comprising 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS).

In accordance with an aspect of the composition, the one or more hydrophilic benefiting ingredients are chosen from skin whitening agents, antioxidants, extracts, antifungal/antibacterial/anti-acne agents, polypeptides, anti-inflammatory agents, keratolytic agents, amino acids, and combinations thereof.

In accordance with an aspect of the composition, the one or more hydrophilic benefiting ingredients are from 40 to 50 wt. % of the total composition.

In accordance with an aspect of the composition, the one or more hydrophilic benefiting ingredients are in a dry solid form at room temperature.

In accordance with an aspect of the composition, the one or more hydrophilic benefiting ingredients have a particle size ranging from 2 to 12μ in diameter.

In accordance with an aspect of the composition, the whitening agents are chosen from Vitamin C, hydroquinone, carbimide peroxide, azelaic acid, cysteine, cysteine HCl, butylhydroquinone, dicetyl, disodium ascorbyl sulfate, erythorbic acid, sugar ester derivatives of Vitamin C, ethyl ferulate, ferulic acid, gallic acid esters, kojic acid, beta & alpha arbutin, Tyrostat (rumex occidentalis extract), cashew fruit extract, curcuma extract, acerola extract, white mulberry moraceae extracts, licorice extract, propyl resorcinol, licorice extract/glabridin, Lactobacillus Ferment Lysate Filtrate, tetrahydrocurcuminoid, and combinations thereof.

In accordance with an aspect of the composition, the Vitamin C is chosen from magnesium ascorbyl phosphate, L-ascorbic acid, ascorbyl glucoside, ascorbic acid, and combinations thereof.

In accordance with an aspect of the composition, the Vitamin C ranges from 40 to 50 wt. % of the composition.

In accordance with an aspect of the composition, the azelaic acid ranges from 0.5 to 10 wt. % of the composition.

In accordance with an aspect of the composition, the antioxidants are chosen from green tea extracts, grape seed extracts, alpha-lipoic acid, potassium sulfite, propyl gallate, quinones, rosmarinic acid, sodium ascorbate, sodium erythorbate, sodium metabisulfite, sodium sulfite, superoxide dismutase, sorbityl furfural, tocopheryl succinate, genistein, quercetin, passiflora extract, chrysin, naringenin-7-glucoside, Vitamin C and combinations thereof.

In accordance with an aspect of the composition, the extracts are chosen from algae extract, aloe barbadensis extract, althea officinalis extract, birch (betula alba) bark extract, borage (borago officinalis) extract, eucalyptus globulus extract, evening primrose (oenothera biennis) extract, geranium maculatum extract, jasmine (jasminum officinale) extract, jojoba (buxus chinensis) oil, kelp, sage (salvia officinalis) extract, licorice extract, uva ursi extract, bearberry extract, rumex crispus extract, blackberry bark extract, gingko biloba extract, ginseng extract, cucumber extracts, willow bark extract and combinations thereof.

In accordance with an aspect of the composition, the antifungal/antibacterial/anti-acne agents are chosen from cedar leaf extract, teat tree extract, citricidal, oleuropein, azelaic acid, Fluorometholone, benzoyl peroxide, Tretinoin, Retinol, Adapalene, Isotretinoin, Motretinide, Clindamycin, Erythromycin, Chloramphenicol, Meclocycline, Resorcinol, aluminium oxide, Dapsone, Vitamin A derivatives, and combinations thereof.

In accordance with an aspect of the composition, the polypeptides are chosen from glutathione (γ-glutamyl-cysteinyl-glycine), copper peptides, palmitoyl entapeptide-4(Matrixyl™) and combinations thereof.

In accordance with an aspect of the composition, the keratolytic agents are chosen from Retin A, topical Fluorouracil, Occlusal-HP (Duoderm) and combinations thereof.

In accordance with an aspect of the composition, the anti-inflammatory agents are chosen from water soluble salts of corticosteroids, noncorticosteroids and combinations thereof.

In accordance with an aspect of the composition, the amino acids are chosen from glycine, lysine, argenine, gluthathione, proline and combinations thereof.

In accordance with an aspect of the composition, the composition is in the form of a stick, a cake, a hot pour or a cream for topical application to skin.

In accordance with an aspect of the composition, the stick, the cake or the hot pour has a melting point of from 40 to 70° C.

In accordance with an aspect of the composition, the cream has a viscosity at room temperature ranging from 5,000 to 300,000 cP.

In accordance with an aspect of the composition, it includes water at less than 5 wt. % of the total composition.

In accordance with an aspect of the composition, the anhydrous hydrophilic/amphiphatic base provides for time-release of the one or more hydrophilic benefiting ingredients after topical application to the skin.

In accordance with an aspect of the composition, further including a humectant chosen from glycerin, sorbitol, xylitol, butylene glycol, polyethylene glycol, propylene glycol and combinations thereof.

In accordance with an aspect of making a dermatological composition for skin and scar treatment includes the steps of: providing components to make a composition comprising 33 to 60 wt. % of one or more hydrophilic benefiting ingredients, and 40 to 60 wt. % of an anhydrous hydrophilic/amphiphatic base, grinding the one or hydrophilic benefiting ingredients in solid form at room temperature to a particle size ranging from 2 to 12μ, making the anhydrous hydrophilic/amphiphatic base by combining 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS) at a temperature ranging from 40 to 60° C., mixing the ground one or more hydrophilic benefiting ingredients into the anhydrous hydrophilic/amphiphatic base at a sufficient shear rate, temperature and time to form a homogenous mixture of the one or more hydrophilic benefiting ingredients, and the anhydrous hydrophilic/amphiphatic base, forming the homogenous mixture into the form of a cake, a stick, a hot pour or a cream, and cooling the formed homogenous mixture to room temperature to form a dermatological composition for topical application to skin.

In accordance with an aspect of making the composition, the mixing and/or forming steps include molding or extruding processes to form the cake, the stick, or the hot pour.

In accordance with an aspect of making the composition, the forming step includes filling the homogenous mixture into a tube for the cream.

In accordance with an aspect of making the composition, the sufficient temperature for mixing is from 22 to 60° C.

In accordance with an aspect of making the composition, the sufficient shear rate for mixing is from 2,000 to 16,000 rpm.

In accordance with an aspect of making the composition, the sufficient time for mixing is from 2 to 60 minutes.

In accordance with an aspect of making the composition, the stick, the cake or the hot pour has a melting point of from 40 to 70° C.

In accordance with an aspect of making the composition, the cream has a viscosity at room temperature ranging from 5,000 to 300,000 cP.

In accordance with an aspect of making the composition, the one or more hydrophilic benefiting ingredients are chosen from skin whitening agents, antioxidants, extracts, antifungal/antibacterial/anti-acne agents, polypeptides, anti-inflammatory agents, keratolytic agents, amino acids and combinations thereof.

In accordance with an aspect of making the composition, the one or more hydrophilic benefiting ingredients are from 40 to 50 wt. % of the total composition.

In accordance with an aspect of making the composition, the one or more hydrophilic benefiting ingredients are in a dry solid form at room temperature.

In accordance with an aspect of making the composition, the one or more hydrophilic benefiting ingredients have a particle size ranging from 2 to 12μ in diameter.

In accordance with an aspect of making the composition, the whitening agents are chosen from Vitamin C, hydroquinone, carbimide peroxide, azelaic acid, cysteine, cysteine HCl, butylhydroquinone, dicetyl, disodium ascorbyl sulfate, erythorbic acid, sugar ester derivatives of Vitamin C, ethyl ferulate, ferulic acid, gallic acid esters, kojic acid, beta & alpha arbutin, Tyrostat (rumex occidentalis extract), cashew fruit extract, curcuma extract, acerola extract, white mulberry moraceae extracts, licorice extract, propyl resorcinol, licorice extract/glabridin, Lactobacillus Ferment Lysate Filtrate, tetrahydrocurcuminoid, and combinations thereof.

In accordance with an aspect of making the composition, the Vitamin C is chosen from magnesium ascorbyl phosphate, L-ascorbic acid, ascorbyl glycoside, ascorbic acid, and combinations thereof.

In accordance with an aspect of making the composition, the Vitamin C ranges from 40 to 50 wt. % of the composition.

In accordance with an aspect of making the composition, the azelaic acid ranges from 0.5 to 10 wt. % of the composition.

In accordance with an aspect of making the composition, the antioxidants are chosen from green tea extracts, grape seed extracts, alpha-lipoic acid, potassium sulfite, propyl gallate, quinones, rosmarinic acid, sodium ascorbate, sodium erythorbate, sodium metabisulfite, sodium sulfite, superoxide dismutase, sorbityl furfural, tocopheryl succinate, genistein, quercetin, passiflora extract, chrysin, naringenin-7-glucoside, Vitamin C and combinations thereof.

In accordance with an aspect of making the composition, the extracts are chosen from algae extract, aloe barbadensis extract, althea officinalis extract, birch (betula alba) bark extract, borage (borago officinalis) extract, eucalyptus globulus extract, evening primrose (oenothera biennis) extract, geranium maculatum extract, jasmine (jasminum officinale) extract, jojoba (buxus chinensis) oil, kelp, sage (salvia officinalis) extract, licorice extract, uva ursi extract, bearberry extract, rumex crispus extract, blackberry bark extract, gingko biloba extract, ginseng extract, cucumber extracts, willow bark extract and combinations thereof.

In accordance with an aspect of making the composition, the antifungal/antibacterial/anti-acne agents are chosen from cedar leaf extract, teat tree extract, citricidal, oleuropein, azelaic acid, Fluorometholone, benzoyl peroxide, Tretinoin, Retinol, Adapalene, Isotretinoin, Motretinide, Clindamycin, Erythromycin, Chloramphenicol, Meclocycline, Resorcinol, aluminium oxide, Dapsone, Vitamin A derivatives, and combinations thereof.

In accordance with an aspect of making the composition, the polypeptides are chosen from glutathione (γ-glutamyl-cysteinyl-glycine), copper peptides, palmitoyl entapeptide-4(Matrixyl™) and combinations thereof.

In accordance with an aspect of making the composition, the keratolytic agents are chosen from Retin A, topical Fluorouracil, Occlusal-HP (Duoderm) and combinations thereof.

In accordance with an aspect of making the composition, the anti-inflammatory agents are chosen from water soluble salts of corticosteroids, noncorticosteroids and combinations thereof.

In accordance with an aspect of making the composition, the amino acids are chosen from glycine, lysine, argenine, gluthathione, proline and combinations thereof.

In accordance with an aspect of making the composition further including water at less than 5 wt. % of the total composition.

In accordance with an aspect of making the composition, the anhydrous hydrophilic/amphiphatic base provides for time-release of the one or more hydrophilic benefiting ingredients after topical application to the skin.

In accordance with an aspect of making the composition further including a humectant chosen from glycerin, sorbitol, xylitol, butylene glycol, polyethylene glycol, propylene glycol and combinations thereof.

In accordance with an aspect of treating skin and scars comprising the steps of: providing a dermatological composition including 33 to 60 wt. % of one or more hydrophilic benefiting ingredients, and 40 to 60 wt. % of an anhydrous hydrophilic/amphiphatic base comprising 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS), and topically applying the dermatological composition to a skin surface area for treatment in a form chosen from a stick, a cream, a cake or a hot pour; and wherein the anhydrous hydrophilic/amphiphatic base provides for time-release of the one or more hydrophilic benefiting ingredients after topically applying the dermatological composition to the skin.

In accordance with an aspect of treating skin and scars, the moisture from skin surface area for treatment initiates time-release of the one or more hydrophilic benefiting ingredients after topically applying the dermatological composition to the skin via a passive release mechanism.

In accordance with an aspect of treating skin and scars further including pre-wetting the skin surface area for treatment prior to topically applying the dermatological composition.

In accordance with an aspect of treating skin and scars, the pre-wet skin surface area for treatment initiates time-release of the one or more hydrophilic benefiting ingredients after topically applying the dermatological composition to the skin via an active release mechanism.

In accordance with an aspect of treating skin and scars further including repeating the topically applying step on at least a daily basis to the skin surface area for treatment.

In accordance with an aspect of treating skin and scars, the skin surface area for treatment undergoes whitening, collagen formation and lightening of scar tissue with time.

In accordance with an aspect of treating skin and scars, the one or more hydrophilic benefiting ingredients are chosen from skin whitening agents, antioxidants, extracts, antifungal/antibacterial/anti-acne agents, polypeptides, anti-inflammatory agents, keratolytic agents, amino acids and combinations thereof.

In accordance with an aspect of treating skin and scars, the one or more hydrophilic benefiting ingredients are from 40 to 50 wt. % of the total composition.

In accordance with an aspect of treating skin and scars, the one or more hydrophilic benefiting ingredients are in a dry solid form at room temperature.

In accordance with an aspect of treating skin and scars, the one or more hydrophilic benefiting ingredients have a particle size ranging from 2 to 12μ in diameter.

In accordance with an aspect of treating skin and scars, whitening agents are chosen from Vitamin C, hydroquinone, carbimide peroxide, azelaic acid, cysteine, cysteine HCl, butylhydroquinone, dicetyl, disodium ascorbyl sulfate, erythorbic acid, sugar ester derivatives of Vitamin C, ethyl ferulate, ferulic acid, gallic acid esters, kojic acid, beta & alpha arbutin, Tyrostat (rumex occidentalis extract), cashew fruit extract, curcuma extract, acerola extract, white mulberry moraceae extracts, licorice extract, propyl resorcinol, licorice extract/glabridin, Lactobacillus Ferment Lysate Filtrate, tetrahydrocurcuminoid, and combinations thereof.

In accordance with an aspect of treating skin and scars, the Vitamin C is chosen from magnesium ascorbyl phosphate, L-ascorbic acid, ascorbyl glycoside, ascorbic acid, and combinations thereof.

In accordance with an aspect of treating skin and scars, the Vitamin C ranges from 40 to 50 wt. % of the composition.

In accordance with an aspect of treating skin and scars, the azelaic acid ranges from 0.5 to 10 wt. % of the composition.

In accordance with an aspect of treating skin and scars, the antioxidants are chosen from green tea extracts, grape seed extracts, alpha-lipoic acid, potassium sulfite, propyl gallate, quinones, rosmarinic acid, sodium ascorbate, sodium erythorbate, sodium metabisulfite, sodium sulfite, superoxide dismutase, sorbityl furfural, tocopheryl succinate, genistein, quercetin, passiflora extract, chrysin, naringenin-7-glucoside, Vitamin C and combinations thereof.

In accordance with an aspect of treating skin and scars, the extracts are chosen from algae extract, aloe barbadensis extract, althea officinalis extract, birch (betula alba) bark extract, borage (borago officinalis) extract, eucalyptus globulus extract, evening primrose (oenothera biennis) extract, geranium maculatum extract, jasmine (jasminum officinale) extract, jojoba (buxus chinensis) oil, kelp, sage (salvia officinalis) extract, licorice extract, uva ursi extract, bearberry extract, rumex crispus extract, blackberry bark extract, gingko biloba extract, ginseng extract, cucumber extracts, willow bark extract and combinations thereof.

In accordance with an aspect of treating skin and scars, the antifungal/antibacterial/anti-acne agents are chosen from cedar leaf extract, teat tree extract, citricidal, oleuropein, azelaic acid, Fluorometholone, benzoyl peroxide, Tretinoin, Retinol, Adapalene, Isotretinoin, Motretinide, Clindamycin, Erythromycin, Chloramphenicol, Meclocycline, Resorcinol, aluminium oxide, Dapsone, Vitamin A derivatives, and combinations thereof.

In accordance with an aspect of treating skin and scars, the polypeptides are chosen from glutathione (γ-glutamyl-cysteinyl-glycine), copper peptides, palmitoyl entapeptide-4(Matrixyl™) and combinations thereof.

In accordance with an aspect of treating skin and scars, the keratolytic agents are chosen from Retin A, topical Fluorouracil, Occlusal-HP (Duoderm) and combinations thereof.

In accordance with an aspect of treating skin and scars, the anti-inflammatory agents are chosen from water soluble salts of corticosteroids, noncorticosteroids and combinations thereof.

In accordance with an aspect of treating skin and scars, the amino acids are chosen from glycine, lysine, argenine, gluthathione, proline and combinations thereof.

In accordance with an aspect of treating skin and scars including water at less than 5 wt. % of the total composition.

In accordance with an aspect of treating skin and scars including a humectant chosen from glycerin, sorbitol, xylitol, butylene glycol, polyethylene glycol, propylene glycol and combinations thereof.

In accordance with an aspect of a dermatological composition for skin and scar treatment comprising, 33 to 60 wt. % of three or more hydrophilic benefiting ingredients including L-ascorbic acid at from 30 to 59.5 wt. %, Lactobacillus Ferment Lysate Filtrate at from 0.1 to 3.0 wt. %, and azelaic acid at from 0.5 to 10.0 wt. %, and 40 to 60 wt. % of an anhydrous hydrophilic/amphiphatic base comprising 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS).

In accordance with an aspect of the dermatological composition for skin and scar treatment, the three or more hydrophilic benefiting ingredients are chosen from skin whitening agents, antioxidants, extracts, antifungal/antibacterial/anti-acne agents, polypeptides, anti-inflammatory agents, keratolytic agents, amino acids and combinations thereof.

In accordance with an aspect of the dermatological composition for skin and scar treatment, the L-ascorbic acid is from 45 to 55 wt. % of the total composition.

In accordance with an aspect of the dermatological composition for skin and scar treatment, the L-ascorbic acid is 50 wt. % of the total composition.

In accordance with an aspect of the dermatological composition for skin and scar treatment, the three or more hydrophilic benefiting ingredients are in a dry solid form at room temperature.

In accordance with an aspect of the dermatological composition for skin and scar treatment, the three or more hydrophilic benefiting ingredients have a particle size ranging from 2 to 12μ in diameter.

In accordance with an aspect of the dermatological composition for skin and scar treatment, the composition is in the form of a stick, a cake, a hot pour or a cream for topical application to skin.

In accordance with an aspect of the dermatological composition for skin and scar treatment, the composition includes water at less than 5 wt. % of the total composition.

In accordance with the dermatological composition for skin and scar treatment, the anhydrous hydrophilic/amphiphatic base provides for time-release of the one or more hydrophilic benefiting ingredients after topical application to the skin.

In accordance with an aspect the dermatological composition for skin and scar treatment, further including a humectant chosen from glycerin, sorbitol, xylitol, butylene glycol, polyethylene glycol, propylene glycol and combinations thereof.

In accordance with an aspect of a dermatological composition for skin and scar treatment comprising, 33 to 60 wt. % of one or more hydrophilic benefiting ingredients, and 40 to 60 wt. % of an anhydrous hydrophilic/amphiphatic base comprising one or more water soluble polyols.

In accordance with an aspect of the dermatological composition for skin and scar treatment, the one or more water soluble polyols are chosen from glycerol, sorbitol, glucose, fructose, lactose, propylene glycol, polyethylene glycol, cetyl alcohol, stearyl alcohol, ethoxydiglycol, dexapanthenol, phenoxyethanol, and tocophersolan (TPGS).

In accordance with an aspect of the dermatological composition for skin and scar treatment, the one or more hydrophilic benefiting ingredients are chosen from skin whitening agents, antioxidants, extracts, antifungal/antibacterial/anti-acne agents, polypeptides, anti-inflammatory agents, keratolytic agents, amino acids, and combinations thereof.

In accordance with an aspect of making a dermatological composition for skin and scar treatment comprising, providing components to make a composition comprising 33 to 60 wt. % of one or more hydrophilic benefiting ingredients, and 40 to 60 wt. % of an anhydrous hydrophilic/amphiphatic base, grinding the one or hydrophilic benefiting ingredients in solid form at room temperature to a particle size ranging from 2 to 12μ, making the anhydrous hydrophilic/amphiphatic base by combining one or more water soluble polyols at a temperature ranging from 40 to 60° C., mixing the ground one or more hydrophilic benefiting ingredients into the anhydrous hydrophilic/amphiphatic base at a sufficient shear rate, temperature and time to form a homogenous mixture of the one or more hydrophilic benefiting ingredients, and the anhydrous hydrophilic/amphiphatic base, forming the homogenous mixture into the form of a cake, a stick, a hot pour or a cream, and cooling the formed homogenous mixture to room temperature to form a dermatological composition for topical application to skin.

In accordance with an aspect of making the composition, the mixing and/or forming steps include molding or extruding processes to form the cake, the stick, or the hot pour.

In accordance with an aspect of making the composition, the forming step includes filling the homogenous mixture into a tube for the cream.

In accordance with an aspect of making the composition, the sufficient temperature for mixing is from 22 to 60° C.

In accordance with an aspect of making a composition, the sufficient shear rate for mixing is from 2,000 to 16,000 rpm.

In accordance with an aspect of making a composition, the sufficient time for mixing is from 2 to 60 minutes.

In accordance with an aspect of making a composition, the one or more hydrophilic benefiting ingredients are chosen from skin whitening agents, antioxidants, extracts, antifungal/antibacterial/anti-acne agents, polypeptides, anti-inflammatory agents, keratolytic agents, amino acids and combinations thereof.

In accordance with an aspect of making a composition, the one or more water soluble polyols are chosen from glycerol, sorbitol, glucose, fructose, lactose, propylene glycol, polyethylene glycol, cetyl alcohol, stearyl alcohol, ethoxydiglycol, dexapanthenol, phenoxyethanol and tocophersolan (TPGS).

In accordance with an aspect of a method of treating skin and scars comprising, providing a dermatological composition including 33 to 60 wt. % of one or more hydrophilic benefiting ingredients, and 40 to 60 wt. % of an anhydrous hydrophilic/amphiphatic base; and topically applying the dermatological composition to a skin surface area for treatment in a form chosen from a stick, a cream, a cake or a hot pour; and wherein the anhydrous hydrophilic/amphiphatic base provides for time-release of the one or more hydrophilic benefiting ingredients after topically applying the dermatological composition to the skin.

In accordance with an aspect of the method of treating skin and scars, the moisture from skin surface area for treatment initiates time-release of the one or more hydrophilic benefiting ingredients after topically applying the dermatological composition to the skin via a passive release mechanism.

In accordance with an aspect of the method of treating skin and scars, including pre-wetting the skin surface area for treatment prior to topically applying the dermatological composition.

In accordance with an aspect of the method of treating skin and scars, the pre-wet skin surface area for treatment initiates time-release of the one or more hydrophilic benefiting ingredients after topically applying the dermatological composition to the skin via an active release mechanism.

In accordance with an aspect of the method of treating skin and scars, including repeating the topically applying step on at least a daily basis to the skin surface area for treatment.

In accordance with an aspect of the method of treating skin and scars, the skin surface area for treatment undergoes whitening, collagen formation and lightening of scar tissue with time.

In accordance with an aspect of the method of treating skin and scars, the one or more hydrophilic benefiting ingredients are chosen from skin whitening agents, antioxidants, extracts, antifungal/antibacterial/anti-acne agents, polypeptides, anti-inflammatory agents, keratolytic agents, amino acids and combinations thereof.

In accordance with an aspect of the method of treating skin and scars, the one or more water soluble polyols are chosen from glycerol, sorbitol, glucose, fructose, lactose, propylene glycol, polyethylene glycol, cetyl alcohol, stearyl alcohol, ethoxydiglycol, dexapanthenol, phenoxyethanol, and tocophersolan (TPGS).

The following are examples of the present disclosure and are not to be construed as limiting.

### EXAMPLES

### Illustrative Example 1

A stable skin and scar treatment composition was produced using the method of making described above and illustrated in FIG. 1 with the following ingredients: Ethoxydiglycol: 10.00 wt. %, Propylene Glycol: 20.00 wt. %, Cetyl Alcohol: 6.00 wt. %, Polyethylene Glycol 1000: 5.00 wt. %, Phenoxyethanol: 0.75 wt. %, Dexapanthenol: 0.75 wt. %, Arbutin: 6.00 wt. %, Lactobacillus Ferment Lysate Filtrate: 1.5 wt. %, Ultrafine Ascorbic Acid 50.00 wt. % for a total of 100.00 wt. %. The composition was effective for skin lightening and whitening and was also stable under normal use conditions.

### Illustrative Example 2

A stable skin and scar treatment composition was produced using the method of making described above and illustrated in FIG. 1 with the following ingredients: Cetyl Alcohol: 10.00 wt. %, Ethoxydiglycol: 4.00 wt. %, Propylene Glycol: 19.00 wt. %, Polyethylene Glycol 1000: 7.00 wt. %, Dexapanthenol: 1.00 wt. %, Azelaic Acid: 3.25 wt. %, Phenoxyethanol: 1.00 wt. %, Tocopherolsolan: 0.75 wt. %, Lactobacillus Ferment Lysate Filtrate: 0.50 wt. %, and Ultrafine Ascorbic Acid: 53.50 wt. % for a total of 100.00 wt. %. The composition was effective for skin lightening and whitening and was also stable under normal use conditions.

### Illustrative Example 3

A stable skin and scar treatment composition was produced using the method of making described above and illustrated in FIG. 1 with the following ingredients: Cetyl Alcohol: 7.75 wt. %, Ethoxydiglycol: 24.0 wt. %, Propylene Glycol: 9.25 wt. %, Polyethylene Glycol 1000: 5.0 wt. %, Dexapanthenol: 1.0 wt. %, Phenoxyethanol: 1.0 wt. %, Tocopherosolan: 1.0 wt. %, Spherical Silica: 5.0 wt. %, Ultrafine Ascorbic Acid: 40.0 wt. %, Tetrahydrocurcuminoids: 5.0 wt. %, and Lactobacillus Ferment Lysate Filtrate: 1.0 wt. % for a total of 100 wt. %. The composition was effective for skin lightening and whitening and was also stable under normal use conditions.

### Illustrative Example 4

A stable skin and scar treatment composition was produced using the method of making as described above and illustrated in FIG. 1 with the following ingredients: Cetyl Alcohol: 7.75 wt. %, Ethoxydiglycol: 20.0 wt. %, Propylene Glycol: 13.25 wt. %, Polyethylene Glycol: 5.00 wt. %, Dexapanthenol: 1.0 wt. %, Phenoxyethanol: 1.0 wt. %, Tocopherosolan: 1.0 wt. %, Spherical Silica: 10.0 wt. %, Ultrafine Ascorbic Acid: 40.0 wt. %, and Lactobacillus Ferment Lysate Filtrate: 1.0 wt. % for a total of 100 wt. %. The composition was effective for skin lightening and whitening and was also stable under normal use conditions.

Applicants have attempted to disclose all embodiments and applications of the disclosed subject matter that could be reasonably foreseen. However, there may be unforeseeable, insubstantial modifications that remain as equivalents. While the present invention has been described in conjunction with specific, exemplary embodiments thereof, it is evident that many alterations, modifications, and variations will be apparent to those skilled in the art in light of the foregoing description without departing from the spirit or scope of the present disclosure. Accordingly, the present disclosure is intended to embrace all such alterations, modifications, and variations of the above detailed description.

All patents, test procedures, and other documents cited herein, including priority documents, are fully incorporated by reference to the extent such disclosure is not inconsistent with this invention and for all jurisdictions in which such incorporation is permitted.

When numerical lower limits and numerical upper limits are listed herein, ranges from any lower limit to any upper limit are contemplated.

What is claimed is:

1. A dermatological composition for skin and scar treatment comprising:
33 to 60 wt. % of one or more hydrophilic benefiting ingredients;
40 to 60 wt. % of an anhydrous hydrophilic/amphiphatic base comprising 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS); and
wherein the composition includes less than 5 wt. % water;
wherein the one or more hydrophilic benefiting ingredients are in a dry solid form at room temperature with a particle size ranging from 2 to 12μ in diameter; and
wherein the anhydrous hydrophilic/amphiphatic base provides for time-release of the one or more hydrophilic benefiting ingredients after topical application to the skin.

2. The composition of claim 1, wherein the one or more hydrophilic benefiting ingredients are chosen from skin whitening agents, antioxidants, extracts, antifungal/antibacterial/anti-acne agents, polypeptides, anti-inflammatory agents, keratolytic agents, amino acids, and combinations thereof.

3. The composition of claim 2, wherein the skin whitening agents are chosen from Vitamin C, hydroquinone, carbimide peroxide, azelaic acid, cysteine, cysteine HCl, butylhydroquinone, dicetyl, disodium ascorbyl sulfate, erythorbic acid, sugar ester derivatives of Vitamin C, ethyl ferulate, ferulic acid, gallic acid esters, kojic acid, beta & alpha arbutin, Tyrostat (rumex occidentalis extract), cashew fruit extract, curcuma extract, acerola extract, white mulberry moraceae extracts, licorice extract, propyl resorcinol, licorice extract/glabridin, Lactobacillus Ferment Lysate Filtrate, tetrahydrocurcuminoid, and combinations thereof.

4. The composition of claim 3, wherein the Vitamin C is chosen from magnesium ascorbyl phosphate, L-ascorbic acid, ascorbyl glucoside, ascorbic acid, and combinations thereof.

5. The composition of claim 4, wherein the Vitamin C ranges from 40 to 50 wt. % of the composition.

6. The composition of claim 2, wherein the polypeptides are chosen from glutathione (γ-glutamyl-cysteinyl-glycine), copper peptides, palmitoyl entapeptide-4(Matrixyl™) and combinations thereof.

7. The composition of claim 1, wherein the composition is in the form of a stick, a cake, a hot pour or a cream for topical application to skin.

8. The composition of claim 7, wherein the stick, the cake or the hot pour has a melting point of from 40 to 70° C.

9. The composition of claim 7, wherein the cream has a viscosity at room temperature ranging from 5,000 to 300,000 cP.

10. The composition of claim 1 further including a humectant chosen from glycerin, sorbitol, xylitol, butylene glycol, polyethylene glycol, propylene glycol and combinations thereof.

11. The composition of claim 1 further including a filler/extender chosen from spherical silica, polyamide, mica, talc, polyethylene, polytetrafluoroethylene, clay, polyester and combinations thereof.

12. A method of making a dermatological composition for skin and scar treatment comprising:
providing components to make a composition comprising 33 to 60 wt. % of one or more hydrophilic benefiting ingredients and 40 to 60 wt. % of an anhydrous hydrophilic/amphiphatic base;
grinding and then mixing the one or hydrophilic benefiting ingredients in a dry solid form at room temperature to a particle size ranging from 2 to 12μ;
making the anhydrous hydrophilic/amphiphatic base by combining 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS) at a temperature ranging from 40 to 60° C.;
mixing the ground and mixed one or more hydrophilic benefiting ingredients into the anhydrous hydrophilic/amphiphatic base at a sufficient shear rate, temperature and time to form a homogenous mixture of the one or more hydrophilic benefiting ingredients, and the anhydrous hydrophilic/amphiphatic base;
forming the homogenous mixture into the form of a cake, a stick, a hot pour or a cream; and
cooling the formed homogenous mixture to room temperature to form a dermatological composition for topical application to skin;
wherein the composition includes less than 5 wt. % water; and
wherein the anhydrous hydrophilic/amphiphatic base provides for time-release of the one or more hydrophilic benefiting ingredients after topical application to the skin.

13. The method of claim 12, wherein the mixing and/or forming steps include molding or extruding processes to form the cake, the stick, or the hot pour.

14. The method of claim 12, wherein the forming step includes filling the homogenous mixture into a tube for the cream.

15. The method of claim 12, wherein the sufficient temperature for mixing is from 22 to 60° C.

16. The method of claim 12, wherein the sufficient shear rate for mixing is from 2,000 to 16,000 rpm.

17. The method of claim 12, wherein the sufficient time for mixing is from 2 to 60 minutes.

18. A method of treating skin and scars comprising:
providing a dermatological composition including 33 to 60 wt. % of one or more hydrophilic benefiting ingredients, and 40 to 60 wt. % of an anhydrous hydrophilic/amphiphatic base comprising 1 to 35 wt. % of propylene glycol, 1 to 15 wt. % of polyethylene glycol, 1 to 20 wt. % of cetyl alcohol, 1 to 20 wt. % stearyl alcohol, 1 to 10 wt. % of ethoxydiglycol, 0.1 to 2.0 wt. % of dexapanthenol, 0.1 to 1.0 wt. % of phenoxyethanol, and 0.1 to 2.0 wt. % of tocophersolan (TPGS); and
topically applying the dermatological composition to a skin surface area for treatment in a form chosen from a stick, a cream, a cake or a hot pour;
wherein the composition includes less than 5 wt. % water;
wherein the one or more hydrophilic benefiting ingredients are in a dry solid form at room temperature with a particle size ranging from 2 to 12μ in diameter; and
wherein the anhydrous hydrophilic/amphiphatic base provides for time-release of the one or more hydrophilic benefiting ingredients after topically applying the dermatological composition to the skin.

19. The method of claim 18, wherein the one or more hydrophilic benefiting ingredients are chosen from skin whitening agents, antioxidants, extracts, antifungal/antibacterial/anti-acne agents, polypeptides, anti-inflammatory agents, keratolytic agents, amino acids, and combinations thereof.

20. A dermatological composition for skin and scar treatment comprising:
33 to 60 wt. % of one or more hydrophilic benefiting ingredients, and
40 to 60 wt. % of an anhydrous hydrophilic/amphiphatic base comprising one or more water soluble polyols, and
wherein the composition includes less than 5 wt. % water,
wherein the one or more hydrophilic benefiting ingredients are in a dry solid form at room temperature with a particle size ranging from 2 to 12μ in diameter, and
wherein the anhydrous hydrophilic/amphiphatic base provides for time-release of the one or more hydrophilic benefiting ingredients after topical application to the skin.

21. The composition of claim 20, wherein the one or more hydrophilic benefiting ingredients are chosen from skin whitening agents, antioxidants, extracts, antifungal/antibacterial/anti-acne agents, polypeptides, anti-inflammatory agents, keratolytic agents, amino acids, and combinations thereof.

22. A method of making a dermatological composition for skin and scar treatment comprising:
providing components to make a composition comprising 33 to 60 wt. % of one or more hydrophilic benefiting ingredients, and 40 to 60 wt. % of an anhydrous hydrophilic/amphiphatic base,
grinding and then mixing the one or hydrophilic benefiting ingredients in dry solid form at room temperature to a particle size ranging from 2 to 12μ,
making the anhydrous hydrophilic/amphiphatic base by combining one or more water soluble polyols at a temperature ranging from 40 to 60° C.,
mixing the ground and mixed one or more hydrophilic benefiting ingredients into the anhydrous hydrophilic/amphiphatic base at a sufficient shear rate, temperature and time to form a homogenous mixture of the one or more hydrophilic benefiting ingredients, and the anhydrous hydrophilic/amphiphatic base,
forming the homogenous mixture into the form of a cake, a stick, a hot pour or a cream, and
cooling the formed homogenous mixture to room temperature to form a dermatological composition for topical application to skin,
wherein the composition includes less than 5 wt. % water, and
wherein the anhydrous hydrophilic/amphiphatic base provides for time-release of the one or more hydrophilic benefiting ingredients after topical application to the skin.

23. The method of claim 22, wherein the one or more hydrophilic benefiting ingredients are chosen from skin whitening agents, antioxidants, extracts, antifungal/antibacterial/anti-acne agents, polypeptides, anti-inflammatory agents, keratolytic agents, amino acids, and combinations thereof.
